(19) United States
(12) Patent Application Publication
(10) Pub. No.: US 2026/0191534 A1
(43) Pub. Date: Jul. 9, 2026

(54) DEVICE AND METHOD FOR REPAIRING A TENDON

(71) Applicant: Pedro Miguel GONÇALVES DINIZ, Lisboa (PT)

(72) Inventor: Pedro Miguel GONÇALVES DINIZ, Lisboa (PT)

(21) Appl. No.: 19/129,010

(22) PCT Filed: Sep. 22, 2023
(86) PCT No.: PCT/IB2023/059406

§ 371 Date: May 11, 2025

(30) Foreign Application Priority Data
Nov. 11, 2022 (PT) 118335
Aug. 18, 2023 (PT) 118875

(51) Int. Cl. A61B 17/11

(52) U.S. Cl. CPC A61B 17/1146; A61B 2017/1103

(57) ABSTRACT

The present disclosure is directed to a device for repairing a tendon comprising two components transversely connected through means for regulating a distance, each component comprising a top portion (2, 4) and a bottom portion (8, 9), each of the bottom portion (8, 9) comprising two substantially parallel structures (10, 11, 14, 15) longitudinally positioned along a central axis (X-X′) of the device, wherein: the means for regulating a distance and the structures (10, 11, 14, 15) are in different planes; each of the structures closer to the axis X-X′ (10, 11) is longer than the structures further from the axis X-X′ (14, 15); and each of the structure (10, 11, 14, 15) comprises at least one aperture along an axis (Z-Z′) and at least one aperture along an axis (W-W′), wherein each aperture in the structure (14) has a correspondent aperture along the axis (Z-Z′) or an axis parallel to (Z-Z′) and in a same plane, in the structure (10, 11, 15).

### FIELD OF THE INVENTION

The present disclosure relates generally to the field of surgical medical devices. More specifically, the disclosure relates to a tendon repair device and method of using the same.

### BACKGROUND OF THE INVENTION

Achilles tendon ruptures (ATRs) are common injuries. This injury's incidence seems to be rising, currently ranging from 6 to 31.17 per 100.000 person-years. These injuries often afflict recreational athletes between the ages of 30 and 40 years and are 3 to 10 times more frequent in men than women. Ruptures of the Achilles tendon are usually located between 2 and 7 cm from the tendon's calcaneal insertion and preceding histopathological tendon changes are common. These injuries may also be caused by laceration or high-energy trauma. Previous studies have shown that early functional rehabilitation provides higher patient satisfaction and improved tendon healing, comparing to immobilization, and that surgical treatment provides improved health-related quality of life and reduces the time until returning to work after injury, comparing to conservative treatment. In addition, clinical data has shown the detrimental effect of complications on long-term outcomes. Namely, patients with re-ruptures or major wound healing complications have persistent functional deficits and worse patient-reported outcomes than patients with uncomplicated primary ruptures. Of note, re-ruptures are more common in patients treated without surgery, i.e., conservatively, and major wound healing complications may occur in patients in which open surgical techniques are used. Hence, many surgeons prefer to employ minimally invasive surgical techniques in the treatment of ATRs. However, biomechanical studies have shown that the loads imparted during early functional rehabilitation may exceed those which minimally invasive techniques are able to withstand, possibly leading to tendon elongation, or even re-rupture. There is fair evidence that post-rupture Achilles tendon elongation may lead to biomechanical changes in the affected limb, which may in turn increase risk of future lower-limb injuries and decreased sports performance.

Patent U.S. Pat. No. 6,200,327B1 discloses an apparatus for taking hold of a tendon comprising an instrument and a needle. The instrument has a pair of inner prongs designed to be inserted inside the peritendinous sheath on either side of the tendon. The instrument also has an outer handle in the form of two outer prongs situated on either side of the inner pair of prongs and in the same plane. These prongs all have orifices on common alignments passing through the tendon. The eye of the needle is of reduced thickness.

However, this device possesses at least two drawbacks. The first is related to the repair stitch employed. Repair techniques employing only sutures placed transversely in the tendon are weak because of the composite material structure of tendons, in which collagen fibers are embedded in a proteoglycan-water matrix and arranged hierarchically and parallel to the tendon's longitudinal axis. Therefore, forcefully pulling the sutures will make them cut through the tendon. Another drawback of this apparatus is related to the placement of the handle. The handle is placed in such a way that blocks access to the tendon through the incision and prevents any other work done in the tendon by other means than the apparatus. Even grasping the tendon and introducing the apparatus can be complicated by impingement of surgical instruments against the distance regulating mechanism by which the two main parts are spread apart.

Utility model CN201775644U discloses an Achilles tendon suture line guiding device, which comprises a fixed guiding block and a sliding guiding block. Guiding arms are arranged on both one side of the fixed guiding block and one side of the sliding guiding block, an Achilles tendon clamping guiding device is formed by the inner guiding arms of the fixed guiding block and the sliding guiding block, a plurality of guiding holes are arranged on the guiding arms, the number of the guiding holes on the guiding arm of the fixed guiding block is the same with that of the guiding holes on the guiding arm of the sliding guiding block, the centers of each front guiding hole and the corresponding rear guiding hole are on the same straight line, two sliding rods are arranged on the fixed guiding block detachably connected with the sliding guiding block by the two sliding rods, and a porous groove sliding along the guiding rods is arranged on the sliding guiding block. In this device, the handle placed at a slope in relation the supports containing the holes for passing the needle. While such handle placement may facilitate the introduction of the apparatus, the difficulties related to obstruction of the access to the tendon in the incision persist and can even be made worse.

As such, considering the foregoing, it may be appreciated that there is a need for novel and improved devices and methods for repairing an Achilles tendon.

Given the importance of avoiding tendon elongation, re-ruptures and wound healing complications while allowing early functional rehabilitation after Achilles tendon ruptures, a device that would allow a stronger means of fixation at the suture tendon interface would be of great interest and benefit for patients. Specifically, a device that would allow the preparation of a locking-loop stitch in a minimally invasive way.

### SUMMARY OF THE INVENTION

In one aspect of the disclosure, there is provided a device for repairing a tendon, preferably an Achilles tendon, comprising two components (100, 200) transversely connected through means for regulating a distance, each component comprising a top portion (2, 4) and a bottom portion (8, 9), each of the bottom portion (8, 9) comprising two substantially parallel structures (10, 11, 14, 15) longitudinally positioned along a central axis (X-X′) of the device, wherein: the means for regulating a distance and the structures (10, 11, 14, 15) are in different planes; each of the structures closer to the axis X-X′ (10, 11) is longer than the structures further from the axis X-X′ (14, 15); and each of the structure (10, 11, 14, 15) comprises at least one aperture along an axis (Z-Z′) and at least one aperture along an axis (W-W′), wherein each aperture in the structure (14) has a correspondent aperture along the axis (Z-Z′) or an axis parallel to (Z-Z′) and in a same plane, in the structure (10, 11, 15).

In a further embodiment, the two components are transversely connected through means for regulating a distance, through the top portion (2, 4).

In a further embodiment, the structures (10, 11) are positioned at an angle (α, α′) between 2° and 10°.

In a further embodiment, a length of the structures (10, 11) is between 4 cm and 15 cm and a length of the structures (14, 15) is between 1 cm and 7 cm.

In a further embodiment, the apertures are circular with a diameter between 1 mm and 5 mm.

In a further embodiment, the apertures of the structures (14, 15) are smaller than the apertures of the structures (10, 11).

In a further embodiment, the apertures of one of the structures (14 or 15) are smaller than the apertures of the structures (10, 11, and 15 or 14).

In a further embodiment, the apertures of each structure (10, 11, 14, 15) are positioned at a distance between 0.5 cm and 3 cm.

In a further embodiment, the means for regulating a distance comprises a distance regulating shaft, a screw locking system, a rack and pinion system, a spring system, lever mechanism system, any combination thereof, or any other means for regulating a distance.

In a further embodiment, the means for regulating a distance comprises a knob for regulating the distance, tiltable butterfly screw, a locking mechanism, a compliant mechanism, or any combination thereof.

In a further embodiment, the device further comprises at least one sliding transversal shaft.

In another aspect of the disclosure, there is provided a system comprising a plurality of devices according to the present disclosure.

In another aspect of the disclosure, there is provided a system comprising the device according to the present disclosure, and means for introduction of a guidewire.

In some embodiments, the means for introduction of a guidewire comprises a rigid cannulated rod, wherein the cannulated rod is substantially curved, angular, or both, and comprises a handle with an aperture in a first extremity and at least two apertures in a second extremity.

In a further embodiment, the handle further comprises a slider.

In a further embodiment, the system further comprises a guidewire, looped-end suture, a needle, or any combination thereof.

In another aspect of the disclosure, there is provided a method for assembly of the device according to the present disclosure, comprising the steps of: a) contacting the two components with the means for regulating a distance, preferably through the top portion (2, 4), wherein at least one sliding transversal shaft is introduced in each of the two components; and b) adding a knob (5), wherein the knob is configured to fix the means for regulating a distance and thereby, allowing the sliding of the two components.

In another aspect of the disclosure, there is provided a use of the device according to the present disclosure, for repairing a partially or totally ruptured tendon, tendon regeneration, repair augmentation, placement of synthetic meshes or scaffolds, placement of biological scaffolds, placement of hydrogels, application of platelet-rich plasma, application of stem cells, application of growth factors, bone-tendon junction repair, or any combination thereof.

In another aspect of the disclosure, there is provided a use of the device according to the present disclosure, in veterinary medicine, laboratory sciences, biomedical engineering, bioengineering, tissue engineering, or any combination thereof.

In another aspect of the disclosure, there is provided a use of the system according to the present disclosure, for repairing a partially or totally ruptured tendon, tendon regeneration, repair augmentation, placement of synthetic meshes or scaffolds, placement of biological scaffolds, placement of hydrogels, application of platelet-rich plasma, application of stem cells, application of growth factors, bone-tendon junction repair, or any combination thereof.

In another aspect of the disclosure, there is provided a use of the system according to the present disclosure, in veterinary medicine, laboratory sciences, biomedical engineering, bioengineering, tissue engineering, or any combination thereof.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present disclosure will become apparent from the detailed description given hereinafter. However, it should be understood that the detailed description and specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

Some embodiments of the disclosure are herein described, by way of example only, with reference to the accompanying drawings. With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

FIGS. 1A-E are a perspective view simplified illustrations of a device and two components, according to some embodiments of the present disclosure;

FIG. 2 is a top view simplified illustration of a device according to some embodiments of the present disclosure;

FIG. 3 is a portion of the top view simplified illustration of a device according to some embodiments of the present disclosure, highlighting the angle (α, α′) of the structure (10, 11);

FIG. 4 is a right-side view simplified illustration of a device according to some embodiments of the present disclosure; and

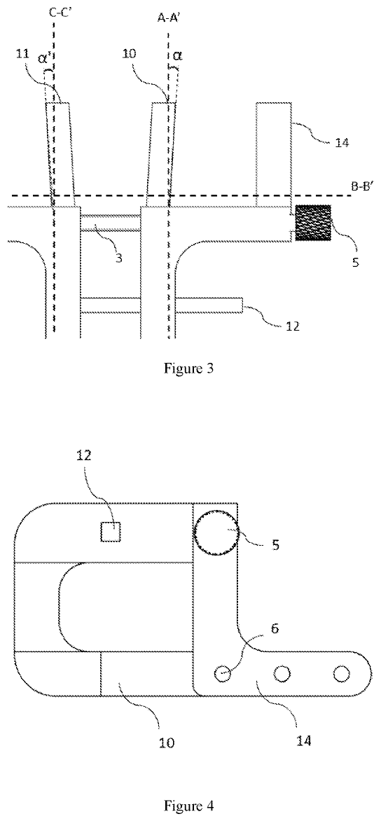

FIGS. 5A-B are perspective view simplified illustrations of a rigid cannulated rod according to some embodiments of the present disclosure.

### DETAILED DESCRIPTION OF THE INVENTION

According to some embodiments, the present disclosure provides a device for repairing a tendon. Non-limiting examples of a tendon according to the present disclosure include an Achilles tendon, quadriceps, patellar, or any combination thereof.

In some embodiments, the device comprises two components, each component comprising a top portion and a bottom portion, both components being transversely connected through means for regulating a distance, and each of the bottom portion comprising two substantially parallel structures longitudinally positioned along a central axis of the device. According to some embodiments, the means for regulating a distance and the two substantially parallel structures are positioned in different planes. According to some embodiments, the means for regulating a distance are positioned in a first plane, and the two substantially parallel structures are positioned in a second plane, wherein the first plane is above the second plane. In some embodiments, the first plane is parallel to the second plane. In some embodiments, the first plane is an angular plane, being at an inclined angle with relation to the second plane. In some embodiments, the second plane is an angular plane, being at an inclined angle with relation to the first plane.

In some embodiments, each of the structures closer to the axis is longer than the structures further from the axis. In some embodiments, each of the structure comprises at least one aperture along an axis wherein each aperture in the structure has a correspondent aperture along the axis Z-Z′ in a correspondent structure.

According to some embodiments, the present disclosure provides a second device, comprising a rigid cannulated rod. In some embodiments, the cannulated rod is substantially curved, angular, or both. In some embodiments, the cannulated rod comprises a handle in a first extremity and at least two apertures in a second extremity.

The present disclosure is based, in part, on the finding that a device as described herein facilitates the access to a tendon through an incision, thereby facilitating its capture by surgical instruments and additional work performed on the tendon, such as passing sutures in planes other than Z-W.

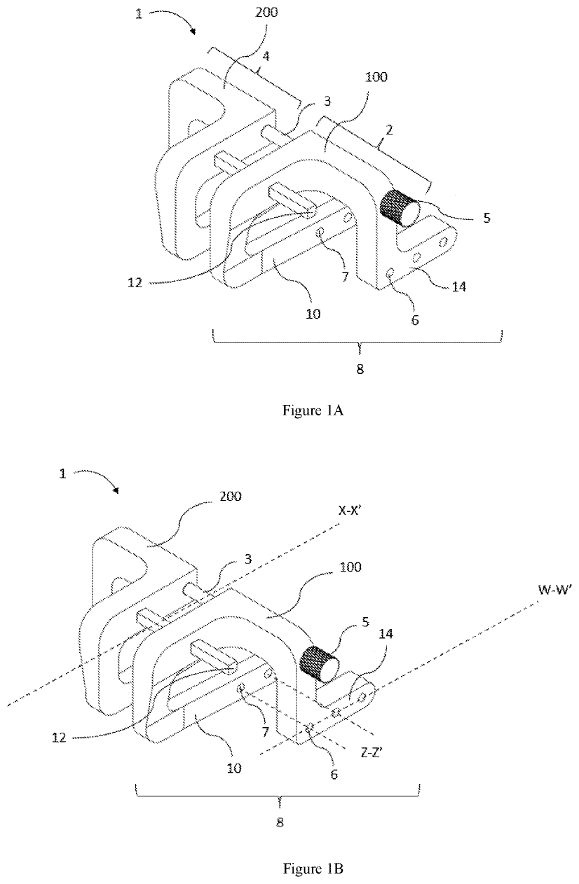

Reference is made to FIGS. 1A-B which are a perspective view simplified illustrations of a device according to some embodiments of the present disclosure.

According to some embodiments, the present disclosure provides a device for repairing a tendon comprising two components (100, 200) transversely connected through means for regulating a distance (3), each component comprising a top portion (2, 4) and a bottom portion (8, 9), each of the bottom portion (8, 9) comprising two substantially parallel structures (10, 11, 14, 15) longitudinally positioned along a central axis (X-X′) of the device, wherein: the means for regulating a distance and the structures (10, 11, 14, 15) are in different planes. In some embodiments, the two components are transversely connected through means for regulating a distance, through the top portion (2, 4).

According to some embodiments, the present disclosure provides a device for repairing an Achilles tendon comprising two components (100, 200) transversely connected through means for regulating a distance, each component comprising a top portion (2, 4) and a bottom portion (8, 9), each of the bottom portion (8, 9) comprising two substantially parallel structures (10, 11, 14, 15) longitudinally positioned along a central axis (X-X′) of the device, wherein: the means for regulating a distance and the structures (10, 11, 14, 15) are in different planes. In some embodiments, the means for regulating a distance is in a plane above and parallel to the plane of the structures (10, 11, 14, 15). In some embodiments, the two components are transversely connected through means for regulating a distance, through the top portion (2, 4).

Reference is made to FIG. 1E which are a perspective view simplified illustration of a device according to some embodiments of the present disclosure.

In a device as described herein, the means for regulating a distance (3) are positioned in a different plane (above and parallel, represented in FIG. 1E by a bright shade) (e.g., in the top portion of each component) from a plane (represented by a dark shade in FIG. 1E) through which the needles and sutures pass [e.g., in the structure (10, 11, 14, 15) in the bottom portion], thereby clearing the access to the tendon. In some embodiments, the means for regulating a distance (3) are positioned in a first plane, the first plane being an angular plane at an inclined angle with relation to a second plane through which the needles and sutures pass [e.g., in the structure (10, 11, 14, 15)). In some embodiments, the second plane is an angular plane, being at an inclined angle with relation to the first plane.

According to the present disclosure a top portion (2,4) and a bottom portion (8,9) are in different planes (represented in FIG. 1E by a bright shade and a dark shade). The top portion (2,4) is positioned in a plane above to the plane of the bottom portion (8,9). In some embodiments, the first plane is parallel to the second plane. In some embodiments, the first plane is an angular plane, being at an inclined angle with relation to the second plane. In some embodiments, the second plane is an angular plane, being at an inclined angle with relation to the first plane.

According to the present disclosure, the means for regulating a distance (3) are positioned in a different plane (above and parallel and represented in FIG. 1E by a bright shade) from a plane of the structure (10, 11, 14, 15), the planes being at a distance (a) between them, wherein the distance (a) is between 5 mm and 30 mm, between 10 mm and 30 mm, or between 10 mm and 20 mm, including any range therebetween. Each possibility represents a separate embodiment of the present disclosure. In some embodiments, the distance (a) is about 15 mm.

The present disclosure is based, in part, on the finding that a device as described herein, enables an improved visualization of a repair site (tendon) and access to the tendon stumps, which in turn allows a repair technique to be performed with greater mechanical resistance. This is due to the fact that the means for regulating a distance (3) is in a plane above and parallel to the plane of the structures (10, 11, 14, 15), and at a distance (a). When the structures (10, 11) are inserted in a skin incision, the means for regulating a distance (3) is in a plane above and parallel to the skin incision and repair site, at a distance (a), therefore it is not in touch with the repair site nor obstructing the vision and access. A distance (a) as described herein represents an optimal distance for allowing a good visualization of the repair site, without compromising the stability of the device.

The present disclosure is based, in part, on the finding that a device as described herein, enables the introduction of a second device (e.g. means for introduction of a guidewire such as a rigid cannulated rod) as described herein. This is possible due to the design of the device, and to the placement of the means for regulating a distance (in the top portion of the device, and in a plane above and parallel to the bottom portion of the device).

In some embodiments, the rod is cannulated to allow the introduction of a flexible guidewire. In some embodiments, the flexible guidewire is a nitinol guidewire, a temporary suture, or any other device or material used to pass sutures through a tendon. In some embodiments, a flexible guidewire as described herein, is used to place a looped-end suture or perform a locking-loop stitch.

The present disclosure is based, in part, on the finding that a device as described herein, enables the use of locked-loop stiches during a repair procedure. In some embodiments, the looped-end sutures are used to retrieve the main sutures through the tendon, creating multiple locking-loop or grasping stitches.

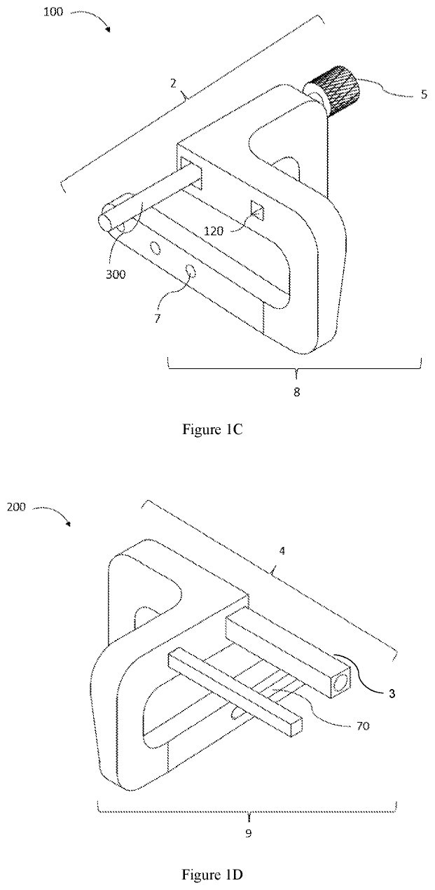

Reference is made to FIGS. 1A-E which are a perspective view simplified illustrations of a device according to some embodiments of the present disclosure, and FIG. 2 which is a top view simplified illustration of a device according to some embodiments of the present disclosure.

According to some embodiments, the present disclosure provides a device for repairing a tendon comprising two components (100, 200), each component comprising a top portion (2, 4) and a bottom portion (8, 9), both components being transversely connected through means for regulating a distance (3), preferably through the top portion (2, 4) and each of the bottom portion (8, 9) comprising two substantially parallel structures (10, 11, 14, 15) longitudinally positioned along a central axis (X-X′) of the device.

According to the present disclosure, the means for regulating a distance (3) is configured to regulate the distance between the two components (100, 200) (by opening and closing) along an axis (Z-Z′).

According to the present disclosure, a means for regulating a distance as described herein is configured to regulate the distance between the structures (10, 11, 14, 15). In some embodiments, a means for regulating a distance is configured to regulate the movement of each one of the structures (10, 11, 14, 15) along an axis (Z-Z′), an axis (W-W′), or both. In some embodiments, a means for regulating a distance regulates the movement of each one of the structures (10, 11, 14, 15) along an axis (Z-Z′). In some embodiments, a means for regulating a distance regulates the movement of at least one of the structures (10, 11, 14, 15) along an axis (Z-Z′).

According to the present disclosure, a means for regulating a distance as described herein is configured to align the structures (10, 11, 14, 15). In some embodiments, a means for regulating a distance regulates the movement of the structures (10, 11, 14, 15) along an axis (W-W′). In some embodiments, a means for regulating a distance regulates the movement of at least one of the structures (10, 11, 14, 15) along an axis (W-W′), until each aperture in one of the structures (10, 11, 14, 15) is aligned with correspondent aperture. In some embodiments, each aperture (6) in the structure (14) comprises a correspondent aperture (7) in the same axis in the structure (10) and a correspondent aperture (70) in the same axis in the structure (11, 15).

Reference is made to FIGS. 1C-D which are a perspective view simplified illustrations of two components (100, 200) according to some embodiments of the present disclosure.

In some embodiments, each of the two components (100, 200) is a single piece comprising all described parts.

In some embodiments, the means for regulating a distance comprises a distance regulating shaft, comprising a screw locking system, a rack and pinion system, a spring system, lever mechanism system, a compliant mechanism, any combination thereof, or any other means for regulating a distance.

In some embodiments, the means for regulating a distance comprises a knob for regulating the distance, a locking mechanism, or both. In some embodiments, the means for regulating a distance (3) comprises a knob (5) for regulating the distance, a locking mechanism, or both. In some embodiments, the means for regulating a distance (3) comprises a complementary part (300).

In some embodiments, the means for regulating a distance (3) comprises a knob (5) connected to the complementary part (300). The complementary part (300) is a transversal shaft that connects to the means for regulating a distance (3). In some embodiments, the transversal shaft that connects to the means for regulating a distance (3), provides improved rigidity to the device.

It should be understood and will become apparent to those skilled in the art, that the means for regulating a distance may assume several forms and shapes. In some embodiments, the means for regulating a distance is made of a rack and pinion mechanism, wherein the rack is placed upwards and proximal to the incision, thus allowing free access to the tendon through this incision. In some embodiments, the rack and pinion mechanism is self-locking and stays in the same position after adjustment. In some embodiments, the device comprises a release button to reduce the distance between the two parts and allow the removal of the device. In some embodiments, the means for regulating a distance is a screwing mechanism. In some embodiments, the means for regulating a distance is a compliant mechanism, and the two components (100, 200) constitute a single piece.

The present disclosure is based, in part, on the finding that the placement of this means for regulating a distance allow unobstructed access to a ruptured end of a tendon. In some embodiments, the means for regulating a distance is placed above a point where the structure (10, 11, 14, 15) intersect a skin incision.

In some embodiments, the device further comprises at least one sliding transversal shaft. In some embodiments, the device further comprises at least one sliding transversal shaft (12). In some embodiments, the device further comprises at least two sliding transversal shafts. According to the present disclosure, a sliding transversal shaft as described herein serves as guidance for sliding, when loosening or tightening the component (100) and component (200).

In some embodiments, the sliding transversal shaft (12) comprises a complementary part (120) to connect component (100) and component (200). In some embodiments, the sliding transversal shaft improves stiffness of the device. In some embodiments, the shape of the sliding transversal shaft (12) is variable. Non-limiting examples of the shape of the sliding transversal shaft (12) include circular, rectangular, and quadrangular. In some embodiments, the shape of the sliding transversal shaft (12) is rectangular. In some embodiments, a sliding transversal shaft (12) with rectangular shape as described herein, increases torsional resistance, when loosening or tightening the component (100) and component (200), therefore giving the device greater resistance and stability.

In some embodiments, the distance between each structures further from the axis X-X′ (14, 15) and the correspondent structures closer to the axis X-X′ (10, 11) is between 1 cm and 5 cm, between 1.2 cm and 5 cm, between 1.5 cm and 5 cm, between 2 cm and 5 cm, between 1 cm and 4 cm, between 1.2 cm and 4 cm, between 1.5 cm and 4 cm, between 2 cm and 4 cm, between 1 cm and 3 cm, between 1.2 cm and 3 cm, between 1.5 cm and 3 cm, or between 2 cm and 3 cm, including any range therebetween. Each possibility represents a separate embodiment of the present disclosure.

In some embodiments, the structures closer to the axis X-X′ (10, 11) are longer than the structures further from the axis X-X′ (14, 15). In some embodiments, the structures closer to the axis X-X′ (10, 11) have a length between 4 cm and 15 cm, between 5 cm and 15 cm, between 4 cm and 8 cm, between 5 cm and 8 cm, between 4 cm and 7 cm, or between 5 cm and 7 cm, including any range therebetween. Each possibility represents a separate embodiment of the present disclosure. In some embodiments, the distance between each structure closer to the axis X-X′ (10, 11) varies according to the opening or closing of the device, controlled by the means for regulating a distance and the knob. This distance can be zero or close to zero, when the device is totally closed to facilitate the introduction in an incision.

In some embodiments, the edges of the structures (10, 11, 14, 15) are substantially curved, thereby allowing for an easier introduction and placement of the device for a repair procedure.

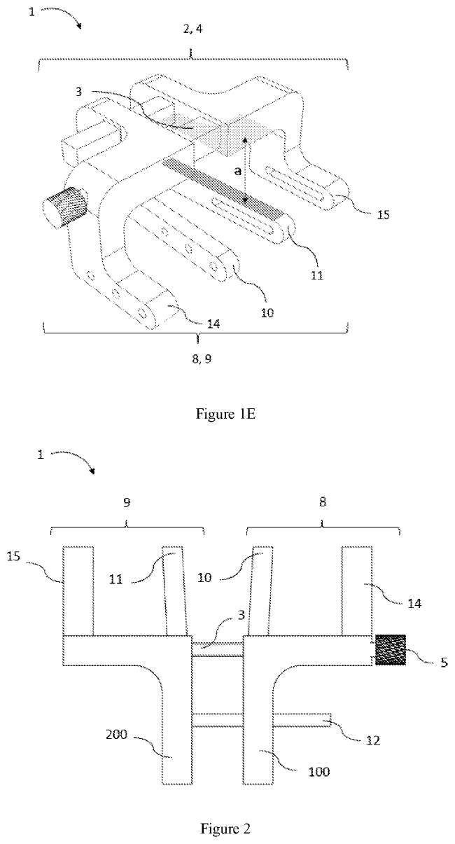

Reference is made to FIG. 2 which is a top view simplified illustration of a device and FIG. 3 which is a portion of the top view simplified illustration of a device according to some embodiments of the present disclosure, highlighting the angle (α, α′) of the structure (10, 11). In some embodiments, the structures closer to the axis X-X′ (10, 11) are positioned at an angled outward towards the structures further from the axis X-X′ (14,15).

In some embodiments, the structures (10, 11) are positioned at an angle (α, α′) between 1° and 15°, between 2° and 15°, between 2° and 10°, between 2° and 8°, between 2° and 7°, between 2° and 6°, between 3° and 10°, between 3° and 8°, between 3° and 7°, between 3° and 6°, between 4° and 10°, between 4° and 8°, between 4° and 7°, or between 4° and 6°, including any range therebetween. Each possibility represents a separate embodiment of the present disclosure. In some embodiments, the structures (10, 11) are positioned at an angle (α, α′) of 5°.

In some embodiments, the angle (α) is defined by the axis A-A′ and B-B′. In some embodiments, the angle (α′) is defined by the axis C-C′ and B-B′.

The present disclosure is based, in part, on the finding that a device as described herein, comprising the structures (10, 11) positioned at an angle (α, α′) as described hereinabove, allows for the tendon to be positioned between the structures (10, 11) without the need to widen an incision.

In some embodiments, the distance between each structure closer to the axis X-X′ (10, 11) is variable.

In some embodiments, the structures further from the axis X-X′ (14, 15) have a length between 1 cm and 10 cm, between 1 cm and 7 cm, between 1 cm and 5 cm, between 2 cm and 7 cm, between 2 cm and 5 cm, between 3 cm and 7 cm, between 3 cm and 5 cm, between 1 cm and 4 cm, between 2 cm and 4 cm, between 3 cm and 4 cm, between 1 cm and 3 cm, or between 2 cm and 3 cm, including any range therebetween. Each possibility represents a separate embodiment of the present disclosure.

In some embodiments, each of the structure (10, 11, 14, 15) comprises at least one aperture along an axis (Z-Z′) wherein each aperture in the structure (10, 11) has a correspondent aperture along the axis Z-Z′ in the structure (14, 15).

In some embodiments, each of the structure (10, 11, 14, 15) comprises at least one aperture along an axis (Z-Z′) wherein each aperture in the structure the structure (14) has a correspondent aperture along the axis (Z-Z′).

In some embodiments, each structure (10, 11, 14, 15) comprises at least one aperture along an axis (W-W′).

In some embodiments, each structure (10, 11, 14, 15) comprises at least two, at least 3, at least 4, at least 5 or at least 10 apertures along an axis (W-W′), including any value therebetween. Each possibility represents a separate embodiment of the present disclosure. It should be understood and will become apparent to those skilled in the art, that the number of apertures is variable and can be adapted according to the needs, and is limited by the length of each structure.

In some embodiments, each structure (10, 11, 14, 15) comprises at least one aperture along an axis (Z-Z′) and at least one aperture along an axis (W-W′), wherein each aperture in the structure (14) has a correspondent aperture along the axis (Z-Z′) or an axis parallel to (Z-Z′) and in a same plane, in the structure (10, 11, 15).

In some embodiments, each aperture (6) in the structure (14) comprises a correspondent aperture (7) in the same axis in the structure (10) and a correspondent aperture (70) in the same axis in the structure (11, 15).

In some embodiments, the apertures are circular with a diameter between 1 mm and 5 mm, between 1 mm and 4.5 mm, between 1 mm and 4 mm, between 1 mm and 3.5 mm, between 1 mm and 3 mm, between 1 mm and 2.9 mm, between 1.5 mm and 5 mm, between 1.5 mm and 4.5 mm, between 1.5 mm and 4 mm, between 1.5 mm and 3.5 mm, between 1.5 mm and 3 mm, between 1.5 mm and 2.9 mm, between 2 mm and 5 mm, between 2 mm and 4.5 mm, between 2 mm and 4 mm, between 2 mm and 3.5 mm, between 2 mm and 3 mm, or between 2 mm and 2.9 mm, including any range therebetween. Each possibility represents a separate embodiment of the present disclosure.

In some embodiments, the apertures of the structures (14, 15) are smaller than the apertures of the structures (10, 11).

In some embodiments, the apertures of one of the structures (14 or 15) are smaller than the apertures of the structures (10, 11, and 15 or 14).

In some embodiments, the apertures of structure (14) are smaller than the apertures of the structures (10, 11, and 15).

In some embodiments, the apertures of structure (15) are smaller than the apertures of the structures (10, 11, and 14).

In some embodiments, the apertures allow for the introduction of a needle.

The present disclosure is based, in part, on the finding that in a device as disclosed herein, a needle might be introduced always through the same side [either through structure (14), or through structure (15)]. Therefore, in one of the structures (14), (15), or both, the apertures are smaller, thereby allowing just enough tolerance to the slide of the needle. In some embodiments, the apertures in the remaining structures are substantially larger than the apertures of the introduction of the needle. In some embodiments, some of the apertures in the remaining structures are merged [please see FIG. 1D, aperture (70)]. The present disclosure is based, in part, on the finding that in a device as disclosed herein, allows for a more straightforward introduction and retrieval of the needle during a repair procedure.

In some embodiments, the apertures (6, 7) of each structure (10, 11, 14, 15) are positioned at a distance between 0.5 cm and 2 cm, between 0.9 cm and 2 cm, between 1 cm and 2 cm, between 0.5 cm and 1.5 cm, between 0.9 cm and 1.5 cm, or between 1 cm and 1.5 cm, between them, including any range therebetween. Each possibility represents a separate embodiment of the present disclosure.

It should be understood and will become apparent to those skilled in the art that all dimensions and sizes of the device and/or any of its constituents as described hereinabove are not an inflexible limitation on the scope of the disclosure. According to the present disclosure, a device and/or any of its constituents may have a different scale, depending on the tendon intended for repair. The scale of a device and/or any of its constituents vary according to the dimension of the tendon intended for repair.

According to some embodiments, the present disclosure provides a use of a device as described hereinabove, for repairing a partially or totally ruptured tendon, tendon regeneration, repair augmentation, placement of synthetic meshes or scaffolds, placement of biological scaffolds, placement of hydrogels, application of platelet-rich plasma, application of stem cells, application of growth factors, bone-tendon junction repair, or any combination thereof.

As used herein, the term “scaffolds” refers to supportive structures that mimic a tendon's extracellular matrix (ECM) and facilitate a healing process. Non-limiting examples of scaffolds include natural extracellular matrix (ECM) scaffolds, decellularized tendon scaffolds, synthetic scaffolds, biodegradable polymers, composite scaffolds, hybrid scaffolds, growth factor-loaded scaffolds, nanofiber scaffolds, hydrogels, and stem cell-seeded scaffolds.

According to some embodiments, the present disclosure provides a use of the device as described hereinabove, in veterinary medicine, laboratory sciences, biomedical engineering, bioengineering, tissue engineering, or any combination thereof.

According to some embodiments, the present disclosure provides a system comprising a plurality of devices as described hereinabove.

According to some embodiments, the present disclosure provides a system comprising a device as described hereinabove, and means for introduction of a guidewire. Several means for introduction of a guidewire are suitable to be used according to the present invention and are known to a skilled person in the art.

In some embodiments, the system comprises at least 2 means for introduction of a guidewire. In some embodiments, the at least 2 means for introduction of a guidewire are connected, preferably with an adjustable distance between them. The technical effect associated is the fact that it allows placing at least two 2 nitinol wires in just one pass and making sure that they are at the same distance from a rupture.

In some embodiments, the means for introduction of a guidewire comprises a rigid cannulated rod. In some embodiments, the means for introduction of a guidewire comprises at least two rigid cannulated rods.

In some embodiments, the cannulated rod is substantially curved, angular, or both. In some embodiments, the cannulated rod comprises a handle in a first extremity and at least two apertures in a second extremity.

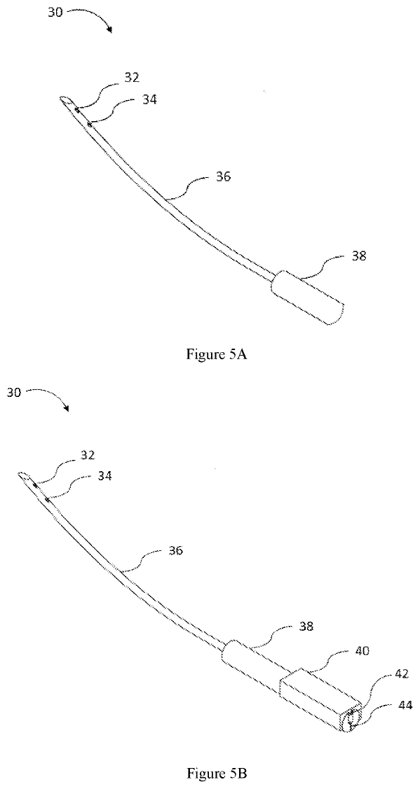

Reference is made to FIGS. 5A-B which are perspective view simplified illustrations of a rigid cannulated rod according to some embodiments of the present disclosure.

In some embodiments, the cannulated rod (30) is substantially curved, angular, or both. In some embodiments, the cannulated rod (30) comprises a handle (38) in a first extremity and at least two apertures (32, 34) in a second extremity. According to the present disclosure, the cannulated rod (30) is made of an appropriate surgical material. Non-limiting examples include surgical stainless steel, titanium, biodegradable polymers, or carbon fiber-reinforced composites. In some embodiments, the cannulated rod (30) is made of surgical stainless steel. In some embodiments, at least two apertures (32,34) are apertures allowing for a guidewire to go through.

In some embodiments, the cannulated rod (30) further comprises a slider (40). In some embodiments, the slider (40) is configured to advance a guidewire.

In some embodiments, the cannulated rod (30) further comprises at least one prong (42, 44). In some embodiments, the at least one prong (42, 44) is a prong for locking a guidewire. In some embodiments, the cannulated rod (30) further comprises a slider (40) and at least one prong (42, 44). In some embodiments, the cannulated rod (30) is cannulated to allow the introduction of a flexible guidewire.

In some embodiments, a guidewire travels through the cannulated rod (30), exits through the aperture (34) closest to the handle (38), and re-enters the cannulated rod (30) through the aperture (32), and is retrieved again in a hole in the handle. In some embodiments, the extremity closer to the aperture (32, 34) is pointed, thereby allowing the piercing of a tendon.

In some embodiments, the cannulated rod (30) has a length between 5 cm and 30 cm, between 10 cm and 30 cm, between 12 cm and 30 cm, between 15 cm and 30 cm, between 20 cm and 30 cm, between 10 cm and 25 cm, between 12 cm and 25 cm, between 15 cm and 25 cm, between 10 cm and 20 cm, between 12 cm and 20 cm, or between 15 cm and 20 cm, including any range therebetween. Each possibility represents a separate embodiment of the present disclosure.

In some embodiments, the cannulated rod (30) has a thickness between 1 mm and 4 mm, between 1 mm and 3 mm, between 1 mm and 2.5 mm, between 1.5 mm and 4 mm, between 1.5 mm and 3 mm, or between 1.5 mm and 2.5 mm, including any range therebetween. Each possibility represents a separate embodiment of the present disclosure.

In some embodiments, the cannulated rod (30) is 16 cm long and has a thickness between 1.5 mm and 2.5 mm (progressively increasing from the one extremity to where it meets the handle), with the handle being 8 cm long and 1.25 cm thick. In some embodiments, the slider is 4.5 cm long and travels 4 cm. In some embodiments, the aperture (32, 34) has a length of 2 mm and spaced 1 cm apart.

In some embodiments, the system further comprises a guidewire, looped-end suture, a needle, or any combination thereof. In some embodiments, the guidewire is a nitinol wire. The nitinol wires are flexible and looped in one end to pass sutures. In some embodiments, the nitinol wires are of different colors, patterns, or any other feature that allows distinction, with the number of nitinol wires matching the number of suture limbs being introduced in each tendon stump in a locking-loop or grasping configuration.

In some embodiments, the needles are straight needles comprising a hole in the blunt end to introduce the sutures. In some embodiments, the needles have a diameter of about 2 mm diameter. In some embodiments, the needles have a length of about 10 cm.

According to some embodiments, the present disclosure provides a method for assembly of the device as described hereinabove, comprising the steps of: a) contacting the two components with the means for regulating a distance, preferably through the top portion (2, 4), wherein a sliding transversal shaft (12) is introduced in each of the two components; and b) adding a knob (5), wherein the knob is configured to fix the means for regulating a distance and thereby, allowing the sliding of the two components.

It should be understood and will become apparent to those skilled in the art that all dimensions and sizes of the system and/or any of its constituents as described hereinabove are not an inflexible limitation on the scope of the disclosure. According to the present disclosure, a system and/or any of its constituents may have a different scale, depending on the tendon intended for repair. The scale of a system and/or any of its constituents vary according to the dimension of the tendon intended for repair.

According to some embodiments, the present disclosure provides a use of the system as described hereinabove, for repairing a partially or totally ruptured tendon, tendon regeneration, repair augmentation, placement of synthetic meshes or scaffolds, placement of biological scaffolds, placement of hydrogels, application of platelet-rich plasma, application of stem cells, application of growth factors, bone-tendon junction repair, or any combination thereof.

According to some embodiments, the present disclosure provides a use of the system as described hereinabove, in veterinary medicine, laboratory sciences, biomedical engineering, bioengineering, tissue engineering, or any combination thereof.

According to some embodiments, the present disclosure provides a device and system for improving the strength of the repair in the treatment of a tendon rupture while keeping a surgery minimally invasive. In some embodiments, a tendon is an Achilles tendon. In some embodiments, a tendon is an Achilles tendon, a quadricep tendon, a patellar tendon, or any combination thereof.

According to some embodiments, the present disclosure, the method of repairing the Achilles tendon using the device (1) as disclosed herein, involves preparing a small surgical incision at a rupture site. First, the paratenon is divided, and access to the ruptured Achilles tendon is attained. Then, the proximal ruptured tendon end is grasped using an appropriate surgical instrument. The device (1) is then introduced through the surgical incision, ensuring that the inner supports [structures (10, 11)] are placed interiorly to the paratenon. The device (1) is then advanced as desired or until the introduction is limited by the surrounding skin while using the means for regulating a distance (3) to move the two components of the device (1) apart. The first needle is then introduced, being usually advisable to introduce the needle through the aperture farthest away from the rupture. A second needle is then introduced in the next aperture. At this point, the cannulated rod (30) is introduced through the incision and anterior to the proximal stump of the ruptured tendon. In some embodiments, the cannulated rod (30) is advanced until the pointed end has passed the needle closest to the surgical wound. Next, the cannulated rod (30) curved end is pressed upwards, in the medial or lateral half of the tendon stump, effectively piercing the tendon while staying below the paratenon. Next, the nitinol wire is advanced at the handle causing it to bulge outside the far end of this apparatus in the space between the tendon and paratenon. The nitinol wire is then retrieved through the incision with the assistance of a Kocher forceps or a hook, taking care to leave the looped end at the desired side of the tendon. For the suture closest to the rupture site the looped-end of the nitinol wire exits anteriorly to the tendon stump, in order to have the suture limbs exit the tendon posteriorly. For the suture in the middle, the looped-end of the nitinol wire exits posteriorly, in order to have the suture limbs exit anteriorly. The cannulated rod (30) is then removed while leaving the nitinol wire in place. The same process is then repeated for the other half of the tendon stump aiming for symmetric placement of the nitinol wires.

In some embodiments, a suture passer is used to pierce the tendon and overlying skin, and advance the nitinol wire percutaneously, with care to avoid injury to the sural nerve. The surgeon may elect to leave the looped-end nitinol wires in place, for easier referencing, before retrieval underneath the paratenon as described above. After placing the first pair of assisting nitinol wires, the main suture is placed on the needle, and the needle is extracted through the other side.

In some embodiments, a pair of suture retrievers, clamps, hooks, carabiners, or any other surgical instruments of deemed appropriate, may be placed on the needle at either side of the tendon and medially to the structures closer to the axis X-X′ (10, 11) before advancing the needle. These instruments can then be used to pull the main suture through the surgical incision without having to remove the device (1), allowing the reintroduction of the main sutures in a locking-loop configuration as described below with the device (1) in place. In some embodiments, the device (1) may come preloaded with suture guides, consisting of looped nitinol wires, nitinol strips with an aperture in the extremity, or any other material, including ferromagnetic materials, in the inner surface of the structures closer to the axis X-X′ (10, 11), through which the needle can travel, that can be pulled or slid outside through the surgical incision to allow the retrieval of a main suture with the device (1) in place. These suture guides may be fixed to the inner surface of the structures closer to the axis X-X′ (10, 11) using snap fit, slot cuts, magnets or any other method deemed appropriate and allow independent extraction of a main suture passing through any of the apertures along the axis (Z-Z′) without having to remove the device. According to some embodiments, these suture guides are pointed in one end and may be used to reintroduce the sutures through the tendon overcoming the need for a suture-passer and nitinol guidewires or temporary sutures.

According to some embodiments, the present disclosure, the process involving the placement of the cannulated rod (30) and pair of nitinol wires is then repeated for remaining aperture in the device (1) while keeping the first needle piercing the tendon to minimize movement of the device (1). The suture farther away from the rupture site can be passed transversely without a locking-loop configuration. After both pairs of nitinol wires have been placed, the device (1) means for regulating a distance (3) is released or moved in the opposite direction, causing the two parts of the device (1) to become nearer and facilitate apparatus removal. The device (1) is then gently removed, pulling the sutures inside the paratenon while keeping them transversely in the tendon. Next, the ends of these sutures are placed in the corresponding pairs of the nitinol wires: the medial thread of the most distant suture is placed in the loop of the medial looped-end suture placed farthest from the rupture site. This process is repeated for all sutures and all suture pairs. The non-looped ends of the nitinol guidewires are then gently pulled in sequence, causing the main sutures to be reintroduced in the tendon through the posterior aspect and exiting at the rupture site, effectively creating a double locking-loop stitch with an additional transverse suture. This process is repeated for the other tendon stump, and sutures are tied sequentially in the corresponding pairs while adjusting tension, starting with the pair the furthest from the rupture site.

According to some embodiments, a device and system as described herein may also be used to perform repairs of quadriceps, patellar, and other tendons, using the same method described hereinabove.

As used herein the term “about” refers to ±10%.

The terms “comprises”, “comprising”, “includes”, “including”, “having” and their conjugates mean “including but not limited to”.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. Device for repairing a tendon, preferably an Achilles tendon, comprising two components (100, 200) transversely connected through means for regulating a distance, each component comprising a top portion (2, 4) and a bottom portion (8, 9), each of said bottom portion (8, 9) comprising two substantially parallel structures (10, 11, 14, 15) longitudinally positioned along a central axis (X-X′) of the device, wherein:
said means for regulating a distance and said structures (10, 11, 14, 15) are in different planes;
each of the structures closer to the axis X-X′ (10, 11) is longer than the structures further from the axis X-X′ (14, 15); and
each of said structure (10, 11, 14, 15) comprises at least one aperture along an axis (Z-Z′) and at least one aperture along an axis (W-W′), wherein each aperture in the structure (14) has a correspondent aperture along the axis (Z-Z′) or an axis parallel to (Z-Z′) and in a same plane, in the structure (10, 11, 15).

2. Device according to claim 1, wherein said two components are transversely connected through means for regulating a distance, through said top portion (2, 4).

3. Device according to claim 1, wherein said structures (10, 11) are positioned at an angle (α, α′) between 2° and 10°.

4. Device according to claim 1, wherein a length of said structures (10, 11) is between 4 cm and 15 cm and a length of said structures (14, 15) is between 1 cm and 7 cm.

5. Device according to claim 1, wherein said apertures are circular with a diameter between 1 mm and 5 mm.

6. Device according to claim 1, wherein said apertures of said structures (14, 15) are smaller than the apertures of said structures (10, 11).

7. Device according to claim 1, wherein said apertures of one of said structures (14 or 15) are smaller than the apertures of said structures (10, 11, and 15 or 14).

8. Device according to claim 1, wherein said apertures of each structure (10, 11, 14, 15) are positioned at a distance between 0.5 cm and 3 cm.

9. Device according to claim 1, wherein said means for regulating a distance comprises a distance regulating shaft, a screw locking system, a rack and pinion system, a spring system, lever mechanism system, or any combination thereof.

10. Device according to claim 1, wherein said means for regulating a distance comprises a knob for regulating the distance, tiltable butterfly screw, a locking mechanism, a compliant mechanism, or any combination thereof.

11. Device according to claim 1, further comprising a sliding transversal shaft.

12. System comprising a plurality of devices according to claim 1.

13. System comprising the device according to claim 1, and means for introduction of a guidewire.

14. System according to claim 13, wherein said means for introduction of a guidewire comprises a rigid cannulated rod, wherein said cannulated rod is substantially curved, angular, or both, and comprises a handle with an aperture in a first extremity and at least two apertures in a second extremity.

15. System according to claim 14, wherein said handle further comprises a slider.

16. System according to claim 12, further comprising a guidewire, looped-end suture, a needle, or any combination thereof.

17. Method for assembly of the device according to claim 1, comprising the steps of:
a) contacting said two components with said means for regulating a distance, preferably through said top portion (2, 4), wherein at least one sliding transversal shaft is introduced in each of said two components; and
b) adding a knob (5), wherein said knob is configured to fix said means for regulating a distance and thereby, allowing the sliding of the two components.

18. Use of the device according to claim 1, for repairing a partially or totally ruptured tendon, tendon regeneration, repair augmentation, placement of synthetic meshes or scaffolds, placement of biological scaffolds, placement of hydrogels, application of platelet-rich plasma, application of stem cells, application of growth factors, bone-tendon junction repair, or any combination thereof.

19. Use of the device according to claim 1, in veterinary medicine, laboratory sciences, biomedical engineering, bioengineering, tissue engineering, or any combination thereof.

20. Use of the system according to claim 12, for repairing a partially or totally ruptured tendon, tendon regeneration, repair augmentation, placement of synthetic meshes or scaffolds, placement of biological scaffolds, placement of hydrogels, application of platelet-rich plasma, application of stem cells, application of growth factors, bone-tendon junction repair, or any combination thereof or in veterinary medicine, laboratory sciences, biomedical engineering, bioengineering, tissue engineering, or any combination thereof.

21. (canceled)
